(19) United States
(12) Patent Application Publication
(10) Pub. No.: US 2005/0032840 A1
(43) Pub. Date: Feb. 10, 2005

(54) Novel therapeutic use of a thienylcyclohexylamine derivative

(76) Inventors: Guy Simonnet, Bordeaux, (FR); Pierre Bernard d'Arbigny, Courbevoie (FR)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/451,055

(22) PCT Filed: Dec. 19, 2001
(86) PCT No.: PCT/FR01/04050

(30) Foreign Application Priority Data
Dec. 20, 2000 (FR) 0016631

(51) Int. Cl. A61K 31/453

(52) U.S. Cl. 514/326

(57) ABSTRACT

The invention concerns the use of a thienylcyclohexylamine, on its own or associated with other substances with pharmaceutical activity, for preparing a medicine designed to prevent and/or treat pain and/or nociception.

The present invention relates to the use of a thienylcyclohexylamine, alone or in combination with other substances with a pharmaceutical activity, for the preparation of a medicament intended to prevent and/or treat pain and/or nociception. The invention also relates to a product comprising a thienylcyclohexylamine and at least one analgesic substance, and a pharmaceutical composition containing it. This product is also particularly useful for preventing the facilitative effects on pain (hyperalgia) paradoxically induced by opiates following their analgesic effect.

By the term pain, should be understood “the disagreeable emotional and sensory experience combined with present or potential tissue damage or described by the patient in such terms” (definition according to the Internal Association for the study of Pain (IASP)). Hereafter, in the present Application, the term pain is used independently in order to designate pain or nociception.

A subject of the invention is therefore the use of thienylcyclohexylamine corresponding to the formula 2-methyl-1-(1-piperidinyl)-1-(2-thienyl)cyclohexane, for the preparation of a medicament intended to prevent and/or treat pain. A particular subject of the invention is the use of thienylcyclohexylamine as defined above, for the treatment of acute pain.

The thienylcyclohexylamine can be used alone or in combination with other substances with a pharmaceutical activity capable of preventing and/or treating pain.

Thienylcyclohexylamine as defined above is described in the Patent EP 396734. Given the existence of 2 asymmetrical carbons, this thienylcyclohexylamine can be in racemic form, or in the form of essentially pure diastereoisomers or enantiomers. The preparation of the diastereoisomers of 1-thienylcyclohexylamine is described in U.S. Pat. No. 5,972,952.

A subject of the invention is also the use of thienylcyclohexylamine as defined above, characterized in that the thienylcyclohexylamine is combined with at least one other substance with a pharmaceutical activity, and preferably with an analgesic. In a very preferable manner, the analgesic is an analgesic acting on the opiate receptors, which is used in strong doses during surgical operations or in repeated manner during the management of intractable or chronic pain. Preferably, the analgesic acting on the opiate receptors is an opiate analgesic.

Among the opiate analgesics commonly used, fentanyl, sufentanil, alfentanil, codeine, pethidine, remifentanil, morphine, tramadol, buprenorphine, nalbuphine, morphine sulphate, hydromorphone hydrochloride, coated morphine sulphate can be mentioned.

A subject of the invention is also a product comprising thienylcyclohexylamine corresponding to the formula 2-methyl-1-(1-piperidinyl)-1-(2-thienyl)cyclohexane in racemic form, or in the form of essentially pure diastereoisomers or enantiomers, and at least one analgesic substance as a combination product for simultaneous or separate use, or use spread out over time in order to treat and/or prevent pain. Preferably, the analgesic is an analgesic acting on the opiate receptors, and highly preferably, the analgesic acting on the opiate receptors is an opiate analgesic.

Preferably, the opiate analgesic combined with the thienylcyclohexylamine is chosen from fentanyl, alfentanil, codeine, pethidine, remifentanyl, morphine, tramadol, buprenorphine, nalbuphine, morphine sulphate, hydromorphone hydrochloride, coated morphine sulphate, and highly preferably the opiate analgesic is fentanyl.

A more particular subject of the invention is, as medicament, a product containing thienylcyclohexylamine as defined above, in racemic form, or in the form of essentially pure diastereoisomers or enantiomers, combined with at least one analgesic substance. A more particular subject of the invention is also a pharmaceutical composition containing, as active ingredient, a medicament as defined above.

The thienylcyclohexylamine as defined above can be administered in a dose comprised between 0.001 and 10 mg/kg, preferably between 0.01 and 1 mg/kg. The substances which are optionally combined with it, such as the opiate analgesic substances, known in pharmacology, are administered in the doses usually advised in the fields of pain and nociception.

The thienylcyclohexylamine as defined above, as well as the substances with a pharmaceutical activity which are optionally combined with it, can be administered by the standard administration routes such as oral, intramuscular, intraperitoneal, subcutaneous or intravenous. They can be administered simultaneously or separately, by identical or different administration routes. Preferably, the thienylcyclohexylamine is administered by intravenous or sub-cutaneous route and the substances with a pharmaceutical activity which are optionally combined with it, such as the analgesic substances, are administered by intravenous or sub-cutaneous route. In the case where the thienylcyclohexylamine is combined with at least one analgesic substance, gacyclidine can be administered before the administration of the analgesic substance.

Finally a subject of the invention is also the use of thienylcyclohexylamine as defined above, for the preparation of a medicament capable of preventing hyperalgias and/or allodynias induced by an analgesic acting on the opiate receptors. In fact, as presented in the experimental part (cf. phase 2), treatment with fentanyl, an opiate analgesic very widely used in hospitals during surgical operations, induces allodynia for several days. This allodynia is completely prevented by the thienylcyclohexylamine according to the invention: a single injection (30 minutes before the analgesic) even at a dose of 0.1 mg/kg which does not per se cause any analgesic effect at this dose, completely prevents this allodynia lasting several days. Moreover, the thienylcyclohexylamine tested does not have any psychomotor effect at the effective doses of 0.1 and 0.3 mg/kg., Preferably the thienylcyclohexylamine is administered before the opiate substance. Also the thienylcyclohexylamine is preferably administered at a dose of less than 5 mg/kg, and very preferably at a dose of less than 0.2 mg/kg.

The administration of 1-[cis-2-methyl-1-(2-thienyl)cyclohexyl]piperidine (gacyclidine), in particular in combination, eases withdrawal, which results in a limit to the duration of hospitalization and a more rapid return to normal life from the professional and social point of view.

The following examples are presented in order to illustrate the above procedures and should in no event be considered as a limit to the scope of the invention.

### EXPERIMENTAL PART

Pharmacological Study

This involved studying, in rats, the effects of 1-[cis-2-methyl-1-(2-thienyl)cyclohexyl]piperidine (gacyclidine), on the immediate analgesic effects of fentanyl but also on the facilitative effects on pain (hyperalgia) induced by the administration of this opiate analgesic carried out in several boluses by venous route in order to approximate to its use in the human surgical clinic.

All the experiments are carried out on Sprague-Dawley rats weighing 350400 g; each experimental phase is carried out according to a similar plan: each experimental phase is carried out on 6 groups of 12 rats including one group of control animals. The test adopted to measure the nociceptive threshold is the Randall-Selitto Test modified according to Kayser et al. (1990) (Kayser V., Basbaum A. I. and Guilbaud G., Deafferentation in the rat increase mechanical nociceptive threshold in the innervated limbs; Brain research (1999), 508, 329-332), using a mechanical stimulus of increasing intensity (expressed in grams), the retained evoked response being the cry of the animal.

The general plan for an experimental phase is as follows:
arrival and housing of the animals: 4 days;
preparation of the animals allowing them to become familiar with the investigator and the experimental conditions and measurements in order to avoid any possibility of measurement bias being induced by stress: 14 days;
preparation and insertion of the catheters: 9 days;
post-operative rest (with antibiotic treatment) and determination of the stability of the experimental measurements (basic nociceptive threshold): 4 days;
testing of response to the different active ingredients: 9 days.

The 3 doses of gacyclidine-retained: 0.1, 0.3 and 1 mg/kg, are injected by venous route using the catheter introduced into a jugular vein.

All the results are analyzed according to an ANOVA test.

Phase 1: Study of the Specific Effects of Gacyclidine on the Nociceptive Threshold in Rats.

Protocol—Phase 1

3 series of experiments each including 2 groups of animals of 12 rats are set up.

1st Series of Experiments:
1st group: the animals receive an injection of physiological saline solution.
2nd group: the animals receive a dose of 0.1 mg/kg of gacyclidine.

2nd Series of Experiments:
1 st group: the animals receive an injection of physiological saline solution.
2nd group: the animals receive a dose of 0.3 mg/kg of gacyclidine.

3rd Series of Experiments:
1st group: the animals receive an injection of physiological saline solution.
2nd group: the animals receive a dose of 1 mg/kg of gacyclidine.

All the animals in the 3 series of experiments are prepared during the same phase. The day of administration of the gacyclidine (or of the physiological saline solution) for each series is deferred by only 3 days in order to follow the evolution of the nociceptive threshold for several days following the administration of the pharmaceutical substances. The measurement of the nociceptive threshold is carried out over at least 4 hours after the injection of gacyclidine, at the rate of one measurement every 30 minutes then daily for at least one week.

Results—Phase 1

The gacyclidine induces an analgesic effect for the first 30 minutes at the 0.3 mg/kg dose, and for the first hour at the 1 mg/kg dose. Interestingly, the gacyclidine only induces motor effects at the strongest dose used (1 mg/kg).

Phase 2: Study of the Ability of Gacyclidine to Counter the Hyperalgia Induced by Naloxone when this is Administered During the Analgesic Effect of Fentanyl.

Protocol—Phase 2

Fentanyl is administered according to a protocol “mimicking” its use in surgery: 4 consecutive intravenous injections (every 15 minutes) of a dose of 40 μg/kg.

Each animal receives 3 types of injection:
the first injection (saline or gacyclidine) is carried out 30 minutes after the measurement of the basic nociceptive threshold,
the series of injections of physiological saline solution or fentanyl (4 consecutive intravenous injections every 15 minutes) is commenced 30 minutes after the first injection of physiological saline solution or gacyclidine,
the third injection (naloxone, the role of which is to block the opiate receptors) is carried out 10 minutes after the last injection of fentanyl; the effect of the naloxone on the nociceptive threshold is assessed 5 minutes after its injection, then at 20 and 35 minutes, then every 30 minutes for two hours.

3 series of experiments each comprising 2 groups of animals of 12 rats are set up.

1st Series of Experiments:
1st group: the animals successively receive physiological saline solution, fentanyl (4×40 μg/kg) then naloxone (1 mg/kg s.c.).
2nd group: the animals successively receive gacyclidine (0.1 mg/kg), fentanyl (4×40 ag/kg) then naloxone (1 mg/kg s.c.).

2nd Series of Experiments:
1st group: the animals successively receive physiological saline solution, fentanyl (4×40 μg/kg) then naloxone (1 mg/kg s.c.).
2nd group: the animals successively receive gacyclidine (0.3 mg/kg), fentanyl (4×40 μg/kg) then naloxone (1 mg/kg s.c.).

3rd Series of Experiments:
1st group: the animals successively receive physiological saline solution, fentanyl (4×40 μg/kg) then naloxone (1 mg/kg s.c.).
2nd group: the animals successively receive gacyclidine (1 mg/kg), fentanyl (4×40 μg/kg) then naloxone (1 mg/kg s.c.).

All the animals in the 3 series of experiments are prepared during the same phase. The day of administration of the pharmaceutical substances for each series is deferred by only 3 days in order to follow the evolution of the nociceptive threshold daily for several days (at least a week) following the administration of these substances, in order to be able to evaluate the amplitude and duration of the hyperalgia induced by the fentanyl. Measurement of the nociceptive threshold is carried out over at least 4 hours after injection of fentanyl, at the rate of one measurement every 30 minutes, the particular effect of the naloxone being measured 5 minutes after the administration of this antagonist of the opiate receptors carried out after the last injection of fentanyl.

Results—Phase 2

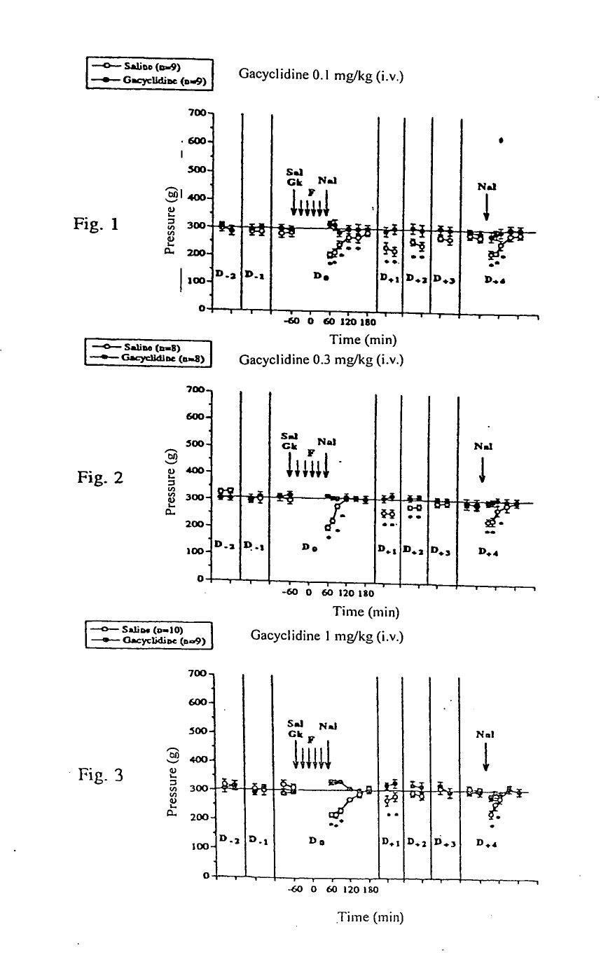

The results are presented in the graphs below (FIGS. 1-3).

Naloxone, injected during the analgesia induced by the fentanyl, causes a considerable lowering of the nociceptive threshold, below the basic values, confirming that the fentanyl activates a nociception facilitator system. This activation is prevented in the animals pre-treated with gacyclidine. It is important to note that even the lowest dose of gacyclidine (0.1 mg/kg) which per se induces neither analgesic effect nor motor effect, already completely prevents this effect.

Phase 3: Evaluation of the Potentiating Effect of Gacyclidine on the Analgesic Effect of Fentanyl.

Fentanyl is administered according to a protocol “mimicking” its use in surgery: 4 consecutive intravenous injections (every 15 minutes) of a dose of 40 μg/kg.

Each animal receives 2 types of injection:
the first injection (saline or gacyclidine) is administered 30 minutes after the measurement of the basic nociceptive threshold,
the series of injections of physiological saline solution or fentanyl (4 consecutive injections of 40 μg/kg of fentanyl every 15 minutes) is commenced 30 minutes after this injection of physiological saline solution or gacyclidine.
   Protocol—Phase 3

3 series of experiments each comprising 2 groups of animals of 12 rats are set up.

1st Series of Experiments:
1st group: the animals successively receive physiological saline solution then fentanyl (4×40 μg/kg).
2nd group: the animals successively receive gacyclidine (0.1 mg/kg) then fentanyl (4×40 μg/kg).

2nd Series of Experiments:
1st group: the animals successively receive physiological saline solution then fentanyl (4×40 μg/kg)
2nd group: the animals successively receive gacyclidine (0.3 mg/kg) then fentanyl (4×40 μg/kg).

3rd Series of Experiments:
1st group: the animals successively receive physiological saline solution then fentanyl (4×40 μg/kg).
2nd group: the animals successively receive gacyclidine (1 mg/kg) then fentanyl (4×40 μg/kg).

All the animals in the 3 series of experiments are prepared during the same phase. The day of administration of the gacyclidine (or of the physiological saline solution) for each series is deferred by only 3 days in order to follow the evolution of the nociceptive threshold (in particular to detect any lowering corresponding to hyperalgia induced by the fentanyl) for several days following the administration of the pharmacological substances. The experimental measurement of the nociceptive threshold is carried out over at least 4 hours after the last injection of fentanyl at the rate of one measurement every 30 minutes then daily for at least one week.

Results—Phase 3

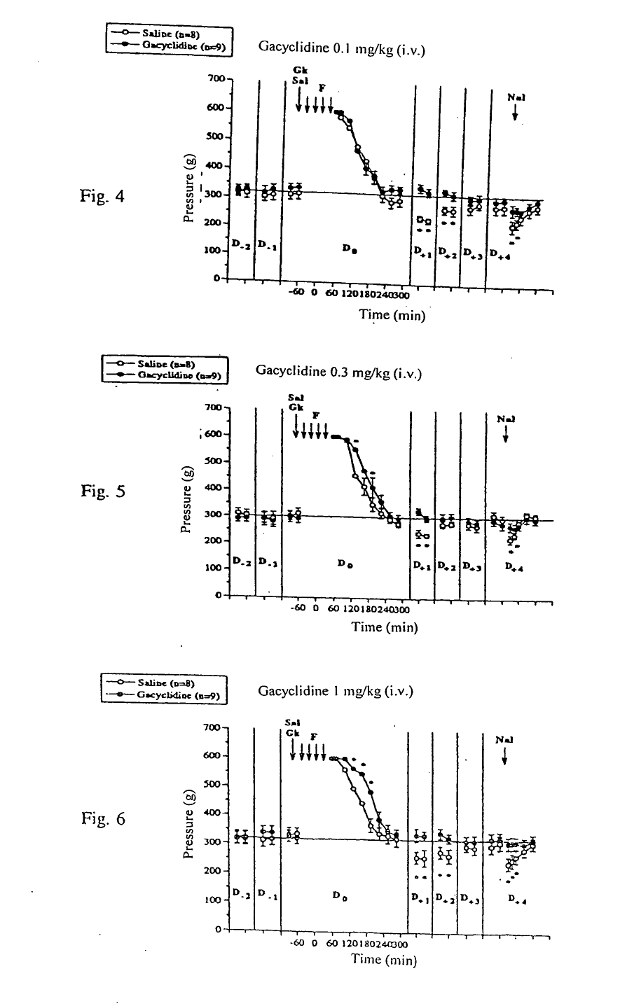

The results are presented in the graphs below (FIGS. 4-6).

At doses of 0.3 and 1 mg/kg, the gacyclidine potentiates the analgesic effect of the fentanyl. At all the doses (0.1, 0.3 and 1 mg/kg), the gacyclidine prevents prolonged allodynia confirming the results of phase 2.

In the graphs below, corresponding to phases 2 and 3, the value of the pressure (expressed in grams) on the animal's paw, the retained response being the cry of the animal, is measured as a function of time (expressed in minutes). For each phase, there are 3 graphs corresponding to the doses of 0.1, 0.3 and 1 mg/kg of gacyclidine respectively.

The abbreviations used are as follows: Sal: saline; GK: gacyclidine; F: fentanyl; Nal: naloxone; *: p<0.05.

1. A method of treating pain in warm-blooded animals comprising administering to warm-blooded animals in need thereof an amount of 2-methyl-1-(1-piperidinyl)-1-(2-thienyl)cyclohexane, in racemic form, or in the form of essentially pure diastereoisomers or enantiomers sufficient to treat pain.

2. The method of claim 1, wherein the pain is acute pain.

3. The method of claim 1 wherein the said compound is combined with at least one other substance with a pharmaceutical activity.

4. The method of claim 3, wherein the said cyclohexane is combined with an analgesic.

5. The method of claim 4, wherein the analgesic acts on the opiate receptors.

6. The method of claim 5, wherein the analgesic acting on the opiate receptors is selected from the group consisting of fentanyl, afentanil, codeine, pethidine, remifentanyl, morphine, tramadol, buprenorphine, nalbuphine, morphine sulfate, hydromorphone hydrochloride and coated morphine sulfate.

7. A composition for treating pain comprising an effective amount of 2-methyl-1-(1-piperidinyl)-1-(2-thienyl)cyclohexane in racemic form, or in the form of essentially pure diastereoisomers or enantiomers, and at least one analgesic sufficient to treat pain.

8. The composition of claim 7 wherein the analgesic acts on the opiate receptors.

9. The composition of claim 8 wherein the analgesic acting on the opiate receptors is selected from the group consisting of fentanyl, alfentanil, codeine, pethidine, remifentanyl, morphine, tramadol, buprenorphine, nalbuphine, morphine sulfate, hydromorphone hydrochloride, coated morphine sulfate.

10. The composition of claim 7 wherein the compound is 1-[cis-2-methyl-1-(2-thienyl)cyclohexyl]piperadine.

11-12.(cancelled).

13. A method of preventing hyperalgias and/or allodynias induced by an analgesic acting on the opiate receptors in warm-blooded animals comprising administering to warm-blooded animals in need thereof an amount of 2-methyl-1-(1-piperidinyl)-1-(2-thienyl) cyclohexane, in racemic form, or in the form of essentially pure diastereoisomers or enantiomers, sufficient to prevent such activity.

14. The method of claim 13, wherein the administration is followed by administration of an opiate substance.

15. The method of claim 13 wherein the said cyclohexane is administered at a dose of less than 5 mg/kg.

16. The method of claim 13 wherein the said cyclohexane is 1-[cis-2-methyl-1-(2-thienyl)cyclohexyl]piperidine.

17. The method of claim 6 wherein the analgesic is fentanyl.

18. The composition of claim 9 wherein the analgesic is fentanyl.
